(19) United States
(12) Patent Application Publication
(10) Pub. No.: US 2026/0345453 A1
(43) Pub. Date: Nov. 5, 2026

(54) ANHYDROUS ANTIPERSPIRANT COMPOSITION

(71) Applicant: Conopco, Inc., d/b/a UNILEVER, Hoboken, NJ (US)

(72) Inventors: Xiaoke Li, Shanghai (CN); Renjiang LIU, Shanghai (CN)

(73) Assignee: Conopco, Inc., d/b/a UNILEVER, Hoboken, NJ (US)

(21) Appl. No.: 19/721,402

(22) Filed: Jun. 26, 2026

Related U.S. Application Data
(63) Continuation of application No. 18/272,379, filed on Jan. 18, 2022.

(30) Foreign Application Priority Data
Feb. 2, 2021 (WO) PCT/CN2021/074753
Mar. 10, 2021 (EU) 21161647.9

(51) Int. Cl. A61K 8/365; A61K 8/31; A61K 8/34; A61K 8/37; A61K 8/49; A61Q 15/00

(52) U.S. Cl. CPC A61K 8/365; A61K 8/31; A61K 8/342; A61K 8/37; A61K 8/4973; A61Q 15/00; A61K 2800/30; A61K 2800/31

(57) ABSTRACT

The present invention is in the field of antiperspirant compositions, in particular, compositions comprising antiperspirant actives. Disclosed is an anhydrous antiperspirant composition comprising (i) 4 to 30% of one or more saturated fatty acid with 14-26 carbons by weight of the composition; (ii) an organic compound selected from alcohols with 8-24 carbons, alkanes with 7-20 carbons, alkyl or alkyl-aryl esters, glyceryl esters, sorbitan esters, ethoxylated sorbitan esters, and a mixture thereof; (iii) 5 to 80% of ethanol by weight of the composition; and (iv) a cosmetically acceptable carrier; wherein said saturated fatty acid is selected from myristic acid, palmitic acid, stearic acid, arachidic acid, behenic acid, lignoceric acid, cerotic acid and 12-Hydroxystearic acid. Also disclosed is a method of reducing perspiration comprising a step of topical application of the anhydrous antiperspirant composition.

### FIELD OF THE INVENTION

The present invention is in the field of antiperspirant compositions.

### BACKGROUND OF THE INVENTION

The present invention relates to compositions that contain antiperspirant actives. These actives are added to compositions to reduce perspiration upon topical application of the compositions to the body, particularly to the underarm regions of the human body viz. the axilla, and sometimes even on the upper part of the body near the chest.

Usually, conventional antiperspirant actives are salts of certain metals having an astringent effect, such as the salts of aluminium and/or zirconium. Since antiperspirants are used regularly, and have been used for decades, there is an ever-increasing need to develop alternative antiperspirant actives which are equally efficacious.

### SUMMARY OF THE INVENTION

The present inventors have surprisingly observed that an anhydrous composition comprising certain amount of specific saturated fatty acid, ethanol and specific organic compound can lead to a reduction in perspiration. The specific saturated fatty acid can be stable in an anhydrous composition with the presence of ethanol and another specific organic solvent. In addition to being stable, the specific saturated fatty acid is capable of forming precipitation/aggregation when they contact with aqueous saline media, which is equivalent to perspiration or sweat, thereby is capable of preventing sweat from rapidly being released. In this manner, such specific saturated fatty acid behave much like conventional antiperspirants. The saturated fatty acid of the present invention should have melting point greater than 37 °C which distinguishes it from other fatty acid.

In accordance with a first aspect is disclosed an antiperspirant composition comprising:
(i) 4 to 30% of one or more saturated fatty acid with 14-26 carbons by weight of the composition;
(ii) an organic compound selected from alcohols with 8-24 carbons, alkanes with 7-20 carbons, alkyl or alkyl-aryl esters, glyceryl esters, sorbitan esters, ethoxylated sorbitan esters, and a mixture thereof;
(iii) 5 to 80% ethanol by weight of the composition; and
(iv) a cosmetically acceptable carrier;
wherein said saturated fatty acid is selected from myristic acid, palmitic acid, stearic acid, arachidic acid, behenic acid, lignoceric acid, cerotic acid and 12-Hydroxystearic acid.

In accordance with a second aspect is disclosed a method of reducing perspiration comprising a step of topical application of the composition of the first aspect on to the desired skin surface.

In accordance with a third aspect is disclosed use of the composition of the first aspect for reduction of bodily perspiration.

All other aspects of the present invention will more readily become apparent upon considering the detailed description and examples which follow.

Except in the examples, or where otherwise explicitly indicated, all numbers in this description indicating amounts of material or conditions of reaction, physical properties of materials and/or use may optionally be understood as modified by the word “about”.

All amounts are by weight of the antiperspirant composition, unless otherwise specified.

It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### DETAILED DESCRIPTION OF THE INVENTION

By “An antiperspirant Composition” as used herein, is meant to include a composition for topical application to the skin of mammals, especially humans. Such a composition is preferably of the leave-on type. By a leave-on composition is meant a composition that is applied to the desired skin surface and left on for one minute to 24 hours after which it may be wiped or rinsed off with water, usually during the regular course of personal washing. The composition may also be formulated into a product which is applied to a human body for improving the appearance, cleansing, odor control or general aesthetics. The composition of the present invention can be in the form of a liquid, lotion, cream, gel or stick form and may be delivered through a roll-on device or using an aerosol can which contains a propellant. “Skin” as used herein is meant to include skin on any part of the body (e.g., neck, chest, back, arms, underarms, hands, legs, buttocks and scalp) especially the underarms.

### Melting Point

“Melting point” refers to the temperature at which the solid and liquid forms of a pure substance can exist in equilibrium. “Melting point” for the purpose of the present invention is specified at a standard pressure such as 1 atmosphere.

### Saturated Fatty Acid

A saturated fatty acid is a type of fatty acid that lacks unsaturated linkages between carbon atoms.

Without wishing to be bound by theory it is believed that the fatty acid has to be in solid form upon application of the composition to the skin, e.g., the underarms. Therefore, it is required that the fatty acid of the present invention has relatively high melting point, have melting point greater than 37 °C.

Antiperspirant compositions of the present invention comprise a saturated fatty acid. The saturated fatty acid of the present invention contains carbon-chains with 14-26 carbons. The saturated fatty acid may have one or more hydroxyl groups.

The saturated fatty acid has a linear hydrocarbon chain.

The saturated fatty acid of the present invention is selected from myristic acid, palmitic acid, stearic acid, arachidic acid, behenic acid, lignoceric acid, cerotic acid, and 12-Hydroxystearic acid. It is more preferred that the saturated fatty acid is selected from stearic acid, palmitic acid, and 12-Hydroxystearic acid.

Antiperspirant compositions in accordance with this invention comprises, from 4 to 30 wt %, and preferably from 4 to 20 wt %, and more preferably from 5 to 15 wt % the saturated fatty acid, based on total weight of the composition.

### Organic Compound

Antiperspirant composition in accordance with this invention comprises an organic compound selected from alcohols with 8-24 carbons, alkanes with 7-20 carbons, alkyl or alkyl-aryl esters, glyceryl esters, sorbitan esters, ethoxylated sorbitan esters, and a mixture thereof. It is preferred that the organic compound is an alkyl or alkyl-aryl ester, alkanes with 7-20 carbons and alcohols with 8-24 carbons. It is more preferred that the organic compound is isopropyl palmitate, isostearyl alcohol or n-dodecane, n-tridecane, n-tetradecane, n-pentadecane, n-hexadecane, isododecane, isohexadecane or a mixture thereof.

It is preferred that the organic compound has a melting point lower than 37 °C, more preferably lower than 20 °C.

Without wishing to be bound by theory it is believed that the organic compound has to be in liquid form to dissolve the saturated fatty acid together with ethanol. Therefore, it is required that the organic compound has relatively low melting point.

When the organic compound is an alcohol with 8-24 carbons, it is preferred to be selected from decyl alcohol, lauryl alcohol, oleyl alcohol and isostearyl alcohol more preferably, it is isostearyl alcohol.

When the organic compound is an alkyl or alkyl-aryl ester, it is preferred to be selected from isopropyl myristate, isopropyl palmitate, myristyl palmitate and C12-15 alkyl benzoate, more preferably, it is isopropyl myristate.

When the organic compound is a glyceryl ester, it is preferred to be selected from glyceryl oleate, glyceryl linoleate, glyceryl linolenate and glyceryl ricinoleate, more preferably it is capric/caprylic triglyceride.

When the organic compound is an alkane with 7-20 carbons, it is preferred to be selected from n-heptane, n-octane, n-nonane, n-decane, n-undecane, n-dodecane, n-tridecane, n-tetradecane, n-pentadecane, n-hexadecane, n-heptadecane, n-octadecane, n-nonadecane, n-eicosane, iso-octane, iso-nonane, iso-decane, iso-undecane, iso-dodecane, iso-tridecane, iso-tetradecane, iso-hexadecane, iso-octadecane, and a mixture thereof. More preferably, it is an alkane with 10-16 carbons, selected from n-decane, n-undecane, n-dodecane, n-tridecane, n-tetradecane, n-pentadecane, n-hexadecane, iso-decane, iso-undecane, iso-dodecane, iso-tridecane, iso-tetradecane, iso-hexadecane, and a mixture thereof, more preferably, it is dodecane.

When the organic compound is a sorbitan ester or ethoxylated sorbitan ester, it is preferred to be selected from Span 20, Span80, Span85, Tween20, Tween 80 and Tween 85, more preferably it is Span 80 or Tween 85.

It is preferred that the antiperspirant compositions in accordance with this invention comprises from 5 to 91 wt %, more preferably from 10 to 60 wt %, furthermore preferably from 15 to 30 wt % of the organic compound, based on total weight of the composition.

Antiperspirant composition in accordance with this invention additionally comprises ethanol. It comprises from 5 wt % to 80 wt %, preferably from 5 to 70 wt %, preferably from 5 to 60 wt %, more preferably from 10% to 50 wt %, furthermore preferably from 30 wt % to 60 wt %, most preferably from 40% to 50 wt %, based on total weight of the composition.

Without wishing to be bound by theory the inventors believe that once the composition is applied, evaporation of the ethanol and contacting with sweat induces the precipitation of saturated fatty acid and which thereby provides antiperspirant benefits.

Antiperspirant compositions in accordance with this invention may advantageously comprise an additional antiperspirant active. However, the composition of the present invention is preferably free from antiperspirant active which comprises aluminium or zirconium. And the composition of the present invention is preferably free from antiperspirant active which is zinc based.

Antiperspirant active which comprises aluminium or zirconium is preferably selected from aluminium/zirconium halides and halohydrate salts, aluminum-zirconium tetrachlorohydrex glycine complex, aluminum-zirconium octachlorohydrex glycine complex, aluminum-zirconium pentachlorohydrate, aluminum sesquichlorohydrate or mixtures thereof, more preferably aluminum sesquichlorohydrate or aluminium chlorohydrates. Antiperspirant active which comprises aluminium or zirconium can also be complexes based on the above-mentioned astringent aluminium and/or zirconium salts and the complex often employs a compound with a carboxylate group, and advantageously this is an amino acid.

Zinc based antiperspirant active is preferably selected from zinc oxide, zinc hydroxide, zinc hydroxide ions with counter ions, and zinc ions with counter ions. Counter ions may include halides and amino acid salt.

Non-aluminum and non-zirconium, non-zinc antiperspirant actives may be present to augment or supplement the antiperspirant activity of the non-thermoplastic polymeric material as disclosed. Non-aluminum or zirconium, or non-zinc antiperspirant active is preferably selected from glycerol monolaurate plus isostearyl alcohol, chitosan or a salt thereof with a weight average molecular weight of from 250 to 650 kDa, titanium compound chelated by alkanolamine with an acid and a polyhydric alcohol, or cholic acid derivative selected from a hydroxycholic acid or a salt thereof with a multivalent metal salt.

More preferably, the composition of the present invention is free from metal based antiperspirant active. The metal includes aluminum, zirconium, zinc, titanium, copper, gallium, stannum, Indium, hafnium, vanadium, cobalt.

### pH of Compositions

It is preferred that pH of the antiperspirant composition of the present invention is preferably from 2 to 9, more preferably 3 to 7.

### Other Ingredients

Other components commonly included in conventional antiperspirant compositions may also be incorporated in the compositions of the present invention. Such components include skin care agents such as emollients, humectants and skin barrier promoters; skin appearance modifiers such as skin lightening agents and skin smoothing agents; anti-microbial agents, in particular organic anti-microbial agents, and preservatives.

The antiperspirant compositions of the invention are applied cosmetically and topically to the skin, broadly speaking, by one of two methods. Different consumers prefer one method or the other. In one method, sometimes called a contact method, the composition is wiped across the surface of the skin, depositing a fraction of the composition as it passes. In the second method, sometimes called the non-contact method, the composition is sprayed from a dispenser held proximate to the skin, often in an area of about 10 to 20 cm². The spray can be developed by mechanical means of generating pressure on the contents of a dispenser, such as a pump or a squeezable sidewall or by internally generated pressure arising from a fraction of a liquefied propellant volatilizing, the dispenser commonly being called an aerosol.

There are broadly speaking two classes of contact compositions, one of which is liquid and usually applied using a roll-on dispenser or possibly absorbed into or onto a wipe, and in the second of which the antiperspirant active is distributed within a carrier liquid that forms a continuous phase that has been gelled. In one variation, the carrier fluid comprises a solvent for the antiperspirant and in a second variation, the antiperspirant remains a particulate solid that is suspended in an oil, usually a blend of oils.

The composition of the invention is anhydrous and comprises cosmetically acceptable carrier. The cosmetically acceptable carrier can be any compound or solvent which is compatible with the other ingredients of the composition. It can be saturated fatty acid or ethanol.

It is preferred that the composition of the invention is in the form of a spray, a firm solid, a soft solid or is a lotion packaged in a roll-on applicator.

It is preferred that, when said composition is a spray it comprises a propellant and the composition is in the form of an aerosol.

### Stick Compositions

In one aspect, the antiperspirant composition of the invention is a stick composition.

Antiperspirant sticks can be formulated as clear (i.e., translucent or transparent) or opaque compositions. Translucent or transparent sticks go on clear and, depending upon their formulation, may remain clear for extended periods of time, reducing the consumer perceived negative of “white marks” associated with deposition of antiperspirant active. Many different materials have been proposed as gellants for a continuous oil phase, including waxes, small molecule gelling agents and polymers. They each have their advantages and of them, one of the most popular class of gellants is waxes, partly at least due to their ready availability and ease of processing, including in particular linear fatty alcohol wax gellants. A gelled antiperspirant composition is applied topically to skin by wiping it across and in contact with the skin, thereby depositing on the skin a thin film.

The nature of the film depends to a significant extent on the gellant that is employed. Although wax fatty alcohols have been employed as gellants for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

Stick compositions are usually available in the form of a firm solid or a soft solid. Firm solids, as the name indicates, are harder and can be directly applied by way of an applicator, for example, to the underarms. Soft solids also need an applicator which is similar to the firm solids, the difference being that the soft solids are softer and the applicator needs to be designed in order to permit extrusion of the solids through a cap member comprising plurality of orifices and the extruded composition can then be applied to the underarms.

### Roll-on

Alternatively, the composition of the invention is a liquid composition, that can be dispensed from a roll-on package. Broadly speaking such compositions could be divided into two classes, namely those in which an antiperspirant active is suspended in a hydrophobic carrier, such as a volatile silicone and those in which the antiperspirant active is dissolved in a carrier liquid, such as glycerin and propylene glycol.

### Aerosol Compositions

Further alternatively, the antiperspirant composition of the invention is delivered through an aerosol composition which comprises a propellant in addition to the applicable other ingredients described hereinabove. Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred.

Propellants herein generally are one of three classes; (i) low boiling-point gasses liquified by compression, (ii) volatile ethers and (iii) compressed non-oxidising gases.

Class (i) is conveniently a low boiling-point material, typically boiling below −5 °C, and often below −15 °C, and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutane. The class (ii) of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The class (iii) of propellant comprises compressed non-oxidising gasses, and in particular carbon dioxide or nitrogen. Inert gases like neon are a theoretical alternative.

The composition of the present invention can comprise a wide range of other optional components. The CTFA Personal Care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: antioxidants, binders, biological additives, buffering agents, colorants, thickeners, polymers, astringents, fragrance, conditioners, exfoliating agents, pH adjusters, other than the ones already discussed earlier, preservatives, natural extracts, essential oils, skin sensates, skin soothing agents, and skin healing agents.

A preservative is a preferred additional component in compositions of the invention. A preservative serves to reduce or eliminate microbial contamination of compositions of the invention. Preservatives are typically employed at a total level of from 0.05 to 3%, preferably at from 0.1 to 2% and most preferably at from 0.4 to 1%.

Suitable preservatives for use with the present invention include 2-phenoxyethanol, polylysine, iodopropynyl butylcarbamate, C₁-C₃ alkyl parabens, sodium benzoate, caprylyl glycol and EDTA. Particularly preferred preservatives are 2-phenoxyethanol, iodopropynyl butylcarbamate, sodium benzoate, caprylyl glycol and EDTA and especially preferred are 2-phenoxyethanol and iodopropynyl butylcarbamate.

A preferred additional component of compositions of the invention is a fragrance. Suitable materials include conventional perfumes, such as perfume oils and also include so-called deo-perfumes, as described in EP 545,556 and other publications. Levels of incorporation are preferably up to 4% by weight, particularly from 0.1% to 2% by weight, and especially from 0.7% to 1.7% by weight.

An antimicrobial deodorant active is a preferred an additional component in compositions of the invention. Such components serve to reduce or eliminate body odour by reducing or otherwise impeding the function of microbes on the skin of the body responsible for malodour generation.

The antimicrobial deodorant active may also be a preservative for the composition.

When employed, the anti-microbial deodorant agent is typically incorporated into the composition at from 0.01% to 3% and particularly at from 0.03% to 0.5%.

Preferred anti-microbial deodorant agents have a minimum inhibitory concentration (MIC) of 1 mg·ml⁻¹ or less, particularly 200 μg·ml⁻¹ or less, and especially 100 μg·ml⁻¹ or less. The MIC of an anti-microbial agent is the minimum concentration of the agent required to significantly inhibit microbial growth. Inhibition is considered “significant” if an 80% or greater reduction in the growth of an inoculum of Staphylococcus epidermidis is observed, relative to a control medium without an anti-microbial agent, over a period of 16 to 24 hours at 37 °C. Details of suitable methods for determining MICs can be found in “Antimicrobial Agents and Susceptibility Testing”, C. Thornsberry, (in “Manual of Clinical Microbiology”, 5^{th} Edition, Ed. A. Balows et al, American Society for Microbiology, Washington D.C., 1991). A particularly suitable method is the Macrobroth Dilution Method as described in Chapter 110 of above publication (pp. 1101-1111) by D. F. Sahm and J. A. Washington II. MICs of anti-microbials suitable for inclusion in the compositions of the invention are triclosan: 0.01-10 μg·ml⁻¹ (J. Regos et al., Dermatologica (1979), 158:72-79) and farnesol: ca. 25 μg·ml⁻¹ (K. Sawano, T. Sato, and R. Hattori, Proceedings of the 17^{th} IFSCC International Conference, Yokahama (1992) p.210-232). By contrast ethanol and similar alkanols have MICs of greater than 1 mg·ml-1.

Suitable organic anti-microbials are bactericides, for example quaternary ammonium compounds, like cetyltrimethylammonium salts; chlorhexidine and salts thereof; and diglycerol monocaprate, diglycerol monolaurate, glycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred anti-microbials for use in the compositions of the invention are polyhexamethylene biguanide salts (also known as polyaminopropyl biguanide salts), an example being Cosmocil CQ™ available from Zeneca PLC, preferably used at up to 1% and more preferably at 0.03% to 0.3% by weight; 2′,4,4′-trichloro, 2-hydroxy-diphenyl ether (triclosan), preferably used at up to 1% by weight of the composition and more preferably at 0.05-0.3%; and 3,7,11-trimethyldodeca-2,6,10-trienol (farnesol), preferably used at up to 1% by weight of the composition and more preferably at up to 0.5%.

Other suitable organic antimicrobial agents are transition metal chelators, as described in WO01/52805, for example. Transitional metal chelators having a binding coefficient for iron (III) of greater than 1026, for example diethylenetriaminepentaacetic acid and salts thereof are preferred.

### Method and Use

The present invention also provides for a method of reducing perspiration comprising a step of topical application of the composition of the first aspect. Preferably, the present invention provides for a method wherein the composition of the first aspect is applied on the underarms. The present invention also provides for a method wherein topical application of the composition of the first aspect reduces perspiration from the surface of the human body. The method in accordance with the invention is preferably non-therapeutic. By non-therapeutic is meant that the method is cosmetic in nature.

The invention also provides for use of the composition of the first aspect for reduction of bodily perspiration. The use in accordance with the invention is preferably non-therapeutic in nature, more preferably cosmetic in nature.

The invention provides for use of the combination of saturated fatty acid with 14-26 carbons, ethanol and organic compound selected from alcohols with 8-24 carbons, alkyl or alkyl-aryl esters, glyceryl esters, sorbitan esters and ethoxylated sorbitan esters as an antiperspirant agent.

The invention will now be demonstrated with the help of the following non-limiting examples.

### EXAMPLES

Materials listed in Table-1 were put through some tests as detailed hereinafter.

TABLE 1

| Trade name | Chemical ingredient | Supplier |
|---|---|---|
| Edenor ST1 MY | stearic acid (55% palmitic | Emery |
| | acid and 45% stearic acid) | |
| 12-Hydroxystearic | 12-hydroxystearic acid | Hallstar |
| Acid | | |
| Prisorine ™ 3515 | isostearyl alcohol | Croda |
| Isopropyl Palmitate | isopropyl palmitate | BASF |
| Span80 | sorbitan monolaurate | TCI Tokoyo |
| Plantasens Flash 80 | dodecane | Clariant |
| Ethanol Absolute | ethanol | Sinopharm |
| | | Chemical |
| | | Reagent (China) |

### Examples

In the following examples all percentages are by weight, unless otherwise indicated.

The compositions indicated in Table-2 were prepared.

TABLE 2

| Reference | | | Etha- |
|---|---|---|---|
| No. | Saturated fatty acid | Organic compound | nol |
| A | 2% stearic acid | 93% isostearyl alcohol | 5% |
| 1 | 4% stearic acid | 91% isostearyl alcohol | 5% |
| 2 | 10% stearic acid | 45% isostearyl alcohol | 45% |
| 3 | 10% stearic acid | 45% isopropyl palmitate | 45% |
| 4 | 10% stearic acid | 45% Span80 | 45% |
| 5 | 20% stearic acid | 30% isostearyl alcohol | 50% |
| 6 | 20% stearic acid | 30% isopropyl palmitate | 50% |
| 7 | 10% 12-hydroxystearic acid | 45% isostearyl alcohol | 45% |
| 8 | 10% 12-hydroxystearic acid | 45% isopropyl palmitate | 45% |
| 9 | 10% 12-hydroxystearic acid | 45% Span80 | 45% |
| 10 | 20% 12-hydroxystearic acid | 30% isostearyl alcohol | 50% |
| 11 | 20% 12-hydroxystearic acid | 30% isopropyl palmitate | 50% |
| 12 | 10% stearic acid | 40% dodecane | 50% |
| 13 | 10% 12-hydroxystearic acid | 40% dodecane | 50% |

The composition of model ionic sweat (pH 6.9) is as given below in Table-3:

TABLE 3

| Ingredient | wt % of total |
|---|---|
| Potassium Chloride | 0.0373 |
| Sodium Bicarbonate | 0.2025 |
| Sodium Chloride | 0.2098 |
| Ammonium Chloride | 0.0107 |
| Calcium Chloride | 0.0222 |
| Lactic Acid | 0.0901 |
| Urea | 0.0018 |
| Water | 99.4256 |

The aggregation/precipitation formation behaviour of the compositions in Table-2 was tested by following the procedure:

1.0 mL of the concerned composition was taken in a vial and mixed with 1.0 mL model ionic sweat. The appearance of each sample was checked visually and recorded.

Results are summarised in Table-4.

TABLE 4

| Reference No. | Appearance |
|---|---|
| A | No Precipitation |
| 1 | Precipitation |
| 2 | Precipitation |
| 3 | Precipitation |
| 4 | Precipitation |
| 5 | Precipitation |
| 6 | Precipitation |
| 7 | Precipitation |
| 8 | Precipitation |
| 9 | Precipitation |
| 10 | Precipitation |
| 11 | Precipitation |
| 12 | Precipitation |
| 13 | Precipitation |

The results (shown in the Table-4) demonstrate that compositions as per the invention (Reference No. 1 to 11) are capable of forming an aggregation/precipitation under the test conditions disclosed earlier, which further indicates their ability to reduce perspiration when used in an antiperspirant composition, while compositions outside the invention (Examples A) do not exhibit such efficacy.

### Stability Test

The compositions indicated in Table-5 were prepared.

TABLE 5

| Reference | | | |
|---|---|---|---|
| No. | Saturated fatty acid | Organic compound | Ethanol |
| B | 4% stearic acid | 96% isostearyl alcohol | 0% |
| C | 4% stearic acid | 95% isostearyl alcohol | 1% |
| D | 4% stearic acid | 94% isostearyl alcohol | 2% |
| 1 | 4% stearic acid | 91% isostearyl alcohol | 5% |
| 14 | 4% stearic acid | 86% isostearyl alcohol | 10% |

The stability of the compositions in Table-5 were tested by following the procedure:

The compositions above were prepared and clear solutions were obtained at room temperature (around 25 °C). Then the samples were stored in fridge at 4 °C overnight (18 hours). Subsequently, the samples were placed under room temperature (around 25 °C) for 24 hours. The appearance of each sample was checked visually and recorded. Clear solution indicates the sample is stable, while the precipitation indicates the sample is unstable.

Results are summarised in Table-6.

TABLE 6

| | |
|---|---|
| Reference | Appearance |

| No. | 4° C. overnight | From 4° C. to room temperature |
|---|---|---|
| B | Precipitation | Precipitation |
| C | Precipitation | Precipitation |
| D | Precipitation | Turns clear |
| 1 | Clear | Clear |
| 14 | Clear | Clear |

The results (shown in the Table-6) demonstrate that compositions as per the invention (Reference No. 1 and 14) are stable under the test conditions disclosed earlier, which, while compositions outside the invention (Examples B to D) do not exhibit such stability.

The compositions indicated in Table-7 were prepared.

TABLE 7

| Reference | | | |
|---|---|---|---|
| No. | Saturated fatty acid | Organic compound | Ethanol |
| 15 | 10% palmitic acid | 30% capric/caprylic | 60% |
| | | triglyceride 70:30 (wt %) | |
| 16 | 10% palmitic acid | 30% Tween 85 | 60% |

The aggregation/precipitation formation behaviour of the compositions in Table-7 was tested by following the procedure:

1.0 mL of the concerned composition was taken in a vial and mixed with 1.0 mL model ionic sweat. The appearance of each sample was checked visually and recorded.

Results are summarised in Table-8.

TABLE 8

| Reference No. | Appearance |
|---|---|
| 15 | Precipitation |
| 16 | Precipitation |

The results (shown in the Table-8) demonstrate that compositions as per the invention (Reference No. 15 to 16) are capable of forming an aggregation/precipitation under the test conditions disclosed earlier, which further indicates their ability to reduce perspiration when used in an antiperspirant composition.

1. An anhydrous antiperspirant composition comprising:
(i) 4 to 30% of one or more saturated fatty acid with 14-26 carbons by weight of the composition;
(ii) an organic compound selected from alcohols with 8-24 carbons, alkanes with 7-20 carbons, alkyl or alkyl-aryl esters, glyceryl esters, sorbitan esters, ethoxylated sorbitan esters, and a mixture thereof;
(iii) 5 to 80% of ethanol by weight of the composition; and
(iv) a cosmetically acceptable carrier; wherein said saturated fatty acid is selected from myristic acid, palmitic acid, stearic acid, arachidic acid, behenic acid, lignoceric acid, cerotic acid and 12-Hydroxystearic acid.

2. An antiperspirant composition as claimed in claim 1 wherein said organic compound has a melting point lower than 37 °C.

3. An antiperspirant composition as claimed in claim 1 or 2 wherein said organic compound is an alcohol selected from decyl alcohol, lauryl alcohol, oleyl alcohol, isostearyl alcohol and a mixture thereof.

4. An antiperspirant composition as claimed in any of claims 1 to 3 wherein said organic compound is an alkane with 7-20 carbon, selected from n-heptane, n-octane, n-nonane, n-decane, n-undecane, n-dodecane, n-tridecane, n-tetradecane, n-pentadecane, n-hexadecane, n-heptadecane, n-octadecane, n-nonadecane, n-eicosane, iso-octane, iso-nonane, iso-decane, iso-undecane, iso-dodecane, iso-tridecane, iso-tetradecane, iso-hexadecane, iso-octadecane and a mixture thereof.

5. An antiperspirant composition as claimed in any of claims 1 to 4 wherein said organic compound is an alkyl or alkyl-aryl ester selected from isopropyl myristate, isopropyl palmitate, myristyl palmitate and C12-15 alkyl benzoate, and a mixture thereof.

6. An antiperspirant composition as claimed in any of claims 1 to 5 wherein said organic compound is a glyceryl ester selected from glyceryl oleate, glyceryl linoleate, glyceryl linolenate and glyceryl ricinoleate, and a mixture thereof.

7. An antiperspirant composition as claimed as any of claims 1 to 6 wherein said organic compound is a sorbitan ester or ethoxylated sorbitan ester selected from Span 20, Span80, Span85, Tween20, Tween 80 and Tween 85, and a mixture thereof.

8. An antiperspirant composition as claimed as any of claims 1 to 7 wherein said composition comprises 5 to 91 wt % of said organic compound, preferably 10 to 60 wt %, more preferably 15 to 30 wt % by weight of the composition.

9. An antiperspirant composition as claimed in any of claims 1 to 8 wherein said composition comprises 4 to 20 wt % of said saturated fatty acid by weight of the composition.

10. An antiperspirant composition as claimed in any of claims 1 to 9 wherein said composition comprises 5 to 60 wt % of ethanol by weight of the composition.

11. An antiperspirant composition as claimed in any of claims 1 to 10 wherein said composition is in the form of a spray, a firm solid, a soft solid or is a lotion packaged in a roll-on applicator.

12. A method of reducing perspiration comprising a step of topical application of the composition as claimed in any of claims 1 to 11 on to the desired skin surface.

13. Use of a composition as claimed in any of claims 1 to 11 for reduction of bodily perspiration.
